(19) United States
(12) Patent Application Publication
(10) Pub. No.: US 2026/0165868 A1
(43) Pub. Date: Jun. 18, 2026

(54) DYNAMIC NECK BRACE

(71) Applicants: Matthew Dhallan, Jackson, WY (US); Kristen Ok, Irvine, CA (US)

(72) Inventors: Matthew Dhallan, Jackson, WY (US); Kristen Ok, Irvine, CA (US)

(21) Appl. No.: 19/529,731

(22) Filed: Feb. 4, 2026

(51) Int. Cl. A61F 5/055

(52) U.S. Cl. CPC A61F 5/055

(57) ABSTRACT

The disclosure relates generally to stabilizing and protecting the neck for individuals subjected to rapid physical forces. More particularly, the disclosure relates to a dynamic neck brace suitable for use for individuals that experience rapid and significant changes in force to the head and neck area.

### CROSS-REFERENCE TO RELATED APPLICATIONS

Not applicable

### FIELD

The disclosure relates generally to stabilizing and protecting the neck for individuals subjected to rapid physical forces. More particularly, the disclosure relates to a dynamic neck brace suitable for use for individuals that experience rapid and significant changes in force to the head and neck area.

### BACKGROUND

High-performance environments, such as aviation, motorsports, and certain industrial settings, often expose individuals to extreme physical forces that can lead to serious injuries. Fighter pilots, for example, experience significant G-forces during rapid maneuvers, which exert pressure on the head and neck. This strain can result in acute injuries and long-term musculoskeletal damage. Existing protective gear, such as helmets and conventional neck supports, primarily focus on impact resistance rather than dynamic load management. These solutions fail to address the continuous and variable forces encountered during high G-force maneuvers, leaving a critical gap in injury prevention.

In addition, individuals, including children, infants, and adults, who are involved in motor vehicle accidents, also can be exposed to physical forces that cause injury. Children often ride in car seats with inadequate head support. Thus, when exposed to an intense physical force, the child's head may move violently. This is particularly true for infants.

Thus, there is a need for an adaptive system that provides real-time support to counteract these forces while maintaining comfort and mobility. The disclosure herein addresses this need by introducing a dynamic neck brace that actively adjusts its support characteristics based on sensor feedback, thereby reducing the risk of neck and spinal injuries without compromising operational performance.

### SUMMARY

In one aspect, the disclosure provides a neck brace. In one aspect, the disclosure provides a neck brace including: one or more support segments, a computing device; and a series of connected cylinders integrated on outer and inner surfaces of the one or more supportive segments, wherein a tension level of the series of connected coils adjusts based on an output from the computing device. In one embodiment, the cylinders are hollow.

In one aspect, the disclosure provides a neck brace including: one or more support segments, a computing device; and a series of connected coils integrated on outer and inner surfaces of the one or more supportive segments, wherein a tension level of the series of connected coils adjusts based on an output from the computing device.

In one aspect, the disclosure provides a neck brace including: one or more support segments, a computing device; and a series of connected tubes integrated on outer and inner surfaces of the one or more supportive segments, wherein a tension level of the series of connected tubes adjusts based on an output from the computing device. In one embodiment, the tubes are hollow tubes.

In one embodiment, the hollow tubes can be individually inflated and deflated with an air compressor. In one embodiment, the hollow tubes can collectively be inflated and deflated with an air compressor. In another embodiment, a subset of the hollow tubes can be inflated and deflated with an air compressor. In one embodiment, stiffness of the tubes can be adjusted by various means including but not limited to via air compressor, pizoelectric material, or any means that can dynamically adjust stiffness in real time rapidly in response to feedback from one or more sensors.

In some embodiments, the computing devices includes a sensor. In some embodiments, the computing device includes a plurality of sensors. In some aspects, the sensors include one or more of: accelerometers, gyroscopes, magnetometers, strain gauge sensors and load cells, electromyography sensors, pulse oximeters, electrocardiogram sensors, vascular pressure sensors, and vibration sensors. In some embodiments, the neck brace contains support segments that include an absorptive padding. In some aspects, the absorptive padding includes one or more of: hexagonal gel support, honeycomb cardboard, aluminum honeycomb, polycarbonate, polypropylene, carbon fiber composite, Kevlar-reinforced polymer, thermoplastic elastomers (TPE), foamed polyurethane, titanium alloy honeycomb, and recycled or non-recycled Polyethylene Terephthalate (PET) honeycomb. In some aspects, the disclosure provides a neck brace, whereby the tension level of a series of connected coils adjusts over multiple supporting segments. In some aspects, the disclosure provides a neck brace, whereby the tension level of the series of connected coils adjusts over a single support segment. In some aspects, the disclosure provides a neck brace, whereby the series of connected coils is a continuous set of coils that is integrated on outer and inner surfaces of the one or more supportive segments. In some aspects the disclosure provides a neck brace, wherein a series of connected coils is a single set of connected coils corresponding to a particular supportive segment, wherein the series of connected coils is integrated on outer and inner surfaces of the supportive segment. In some embodiments, the disclosure provides a neck brace, wherein the computing device of the neck brace further includes: a memory; a processor communicatively coupled to the memory; wherein the memory stores a set of instructions, which, when executed by the processor, cause the processor to: receive data from the sensor; determine an ideal coil tension setting for a user based on the sensor data using a Force machine learning model; provide a result of ideal coil tension settings; compare the result of the ideal coil tension settings with real-time sensor data; determine if a tension level of coils needs to be adjusted; and output a signal to adjust the tension level of the series of connected coils. In some embodiments, the memory further includes a set of instructions to generate training datasets based on ideal coil tension settings for a user. In some aspects, the disclosure provides a neck brace further including a cloth.

In one aspect, the disclosure provides a system for protecting the head or neck from injury, the system including: a neck brace including one or more support segments; a series of connected coils integrated on outer and inner surfaces of the supportive segment; a computing device including: a sensors; a memory; a processor communicatively coupled to the memory; wherein the memory stores a set of instructions, which, when executed by the processor, cause the processor to: receive data from the sensor; determine an ideal coil tension setting for a user based on the sensor data using a Force machine learning model; provide a result of ideal coil tension settings; compare the result of the ideal coil tension settings with real-time sensor data; determine if a tension level of coils needs to be adjusted; and output a signal to adjust the tension level of the series of connected coils. In some embodiments, the disclosure of the system includes one or more support segments with an absorptive padding. In some aspects, the absorptive padding includes one or more of a: hexagonal gel support, honeycomb cardboard, aluminum honeycomb, polycarbonate, polypropylene, carbon fiber composite, Kevlar-reinforced polymer, thermoplastic elastomers (TPE), foamed polyurethane, titanium alloy honeycomb, and recycled or non-recycled Polyethylene Terephthalate (PET) honeycomb. In some aspects, the disclosure provides a system, wherein a tension level of the series of connected coils adjusts over multiple supporting segments. In some aspects, the disclosure provides a system, wherein a tension level of the series of connected coils adjusts over a single support segment. In some aspects, the disclosure proves a system, wherein a series of connected coils is a continuous set of coils that is integrated on outer and inner surfaces of the one or more supportive segments. In some aspects, the disclosures provides a system further including a cloth.

These and other aspects of the disclosure will become more fully understood upon a review of the drawings and the detailed description, which follows. Other aspects, features, and embodiments of the disclosure will become apparent to those skilled in the art, upon reviewing the following description of specific, example embodiments of the disclosure in conjunction with the accompanying figures. While features of the disclosure may be discussed relative to certain embodiments and figures below, all embodiments of the disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 illustrates aspects of the dynamic neck brace, according to some embodiments.

FIG. 2 illustrates a top-view of a support segment, according to some embodiments.

FIG. 3 illustrates a front-view of the dynamic neck brace, according to some embodiments.

FIG. 4 illustrates an example of the neck brace attached to a helmet, according to some embodiments.

FIG. 5 illustrates a depiction of components of a computing device, according to some embodiments.

FIG. 6 illustrates aspects of the dynamic neck brace with a compressed coil over multiple supporting segments, according to some embodiments.

FIG. 7 illustrates aspects of the dynamic neck brace with a compressed coil over a single supporting segment, according to some embodiments.

FIG. 8 illustrates an example of the neck brace attached to a helmet in response to a force, according to some embodiments.

FIG. 9 illustrates aspects of the dynamic neck brace, according to some embodiments.

FIG. 10 illustrates aspects of the dynamic neck brace with a compressed coil, according to some embodiments.

FIG. 11 illustrates an example of the neck brace attached to a helmet in response to a force, according to some embodiments.

FIG. 12 illustrates aspects of the dynamic neck brace with multiple support segments containing compressed coils, according to some embodiments.

FIG. 13 is a block diagram conceptually illustrating a system where a central computer of the aircraft or vehicle can communicate with the neck brace, according to some embodiments.

FIG. 14 is a flowchart illustrating an example process for outputting a signal to adjust the tension of the coil system, according to some embodiments.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the disclosure. However, it will be apparent to those skilled in the art that the various features, concepts and embodiments described herein may be implemented and practiced without these specific details. In some instances, well-known structures and components are shown in block diagram form to avoid obscuring such concepts.

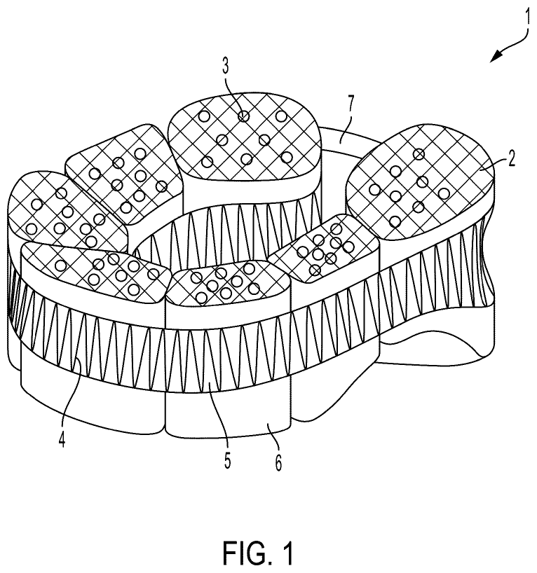

Referring to FIG. 1, neck brace 1 is depicted from an orthogonal view. In some embodiments, neck brace 1 comprises one or more support segments 6, a computing device 3, and a series of connected coils 4 integrated on outer and inner surfaces of the one or more supportive segments. In some embodiments, the series of connected coils 4 is a continuous set of coils that surrounds the one or more support segments 6. In some embodiments, the continuous set of coils is a series of connected coils 4 connected as a single unit across multiple support segments. In some embodiments, neck brace 1 contains a single computing device. In other embodiments, the neck brace contains 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 computing devices.

In some embodiments, neck brace 1 is comprised of one support segment 6. In other embodiments, the neck brace 1 contains 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 support segments. In some embodiments, the support segments 6 are an approximate cylindrical shape (as depicted in FIG. 1). In other embodiments, the support segments 6 may be rectangular or square shapes. In other embodiments, the support segments 6 may be curved or contoured shapes and may conform to the anatomy of the neck and shoulders of the user.

In some embodiments, the support segments 6 may be fabricated from a range of materials selected for their strength, durability, and ergonomic performance. For instance, the support segment 6 may be made from carbon fiber composites, aluminum alloys, polycarbonate; or Kevlar-reinforced polymers. In other embodiments, the support segments 6 may be made from thermoplastic elastomers (TPE), foamed polyurethane, titanium alloys, and/or recycled or non-recycled polyethylene terephthalate.

In some embodiments, neck brace 1 may be covered by a cloth. For instance, in some embodiments, the cloth may comprise a woven mesh fabric formed from synthetic fibers such as polyester or nylon, providing high tensile strength while allowing air circulation through interlaced openings. In other embodiments, the cloth may include a laminated composite structure with a microporous membrane bonded to a lightweight textile layer, enabling moisture vapor transmission while maintaining water resistance. In additional embodiments, the cloth may utilize knitted spacer fabrics incorporating three-dimensional loops that create air channels for enhanced ventilation and cushioning. In further examples, the cloth may be constructed from natural fibers, such as cotton or bamboo, blended with elastomeric threads to improve flexibility and comfort.

In some embodiments, the series of connected coils 4 are made from spring steel. In other embodiments, the series of connected coils 4 can be made from titanium alloy. In other examples, the series of connected coils 4 can be made from carbon fiber composite. In further embodiments, the series of connected coils 4 can be made from shape-memory alloys such as Nitinol. In additional embodiments, the series of connected coils 4 can be made from high-performance polymers like polyether ether ketone (PEEK).

In some embodiments, the material 5 surrounding the series of connected coils 4 can be made from a sheet of flexible metal material. For example, the material 5 could be a sheet of gold, silver, copper, aluminum, iron, nickel or titanium.

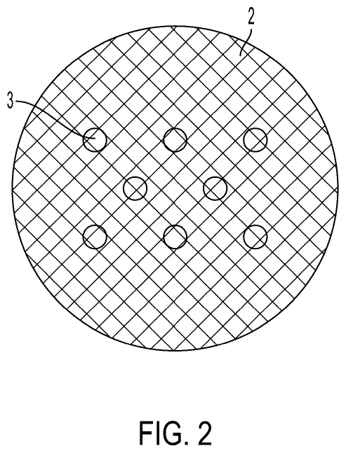

Referring to FIG. 2, a single support segment is depicted from a top view. In some embodiments, the absorptive padding 2 is made from a hexagonal gel support. In some examples, the hexagonal gel refers to a cushioning or supportive material that is structured in a honeycomb-like pattern, where the cells are hexagonal in shape and filled or formed from a gel substance. In other embodiments, the absorptive padding 2 can be made from one of or at least one of: honeycomb cardboard, aluminum honeycomb, polycarbonate, polypropylene, carbon fiber composite, Kevlar-reinforced polymer, thermoplastic elastomers (TPE), foamed polyurethane, titanium alloy honeycomb, and recycled or non-recycled Polyethylene Terephthalate (PET) honeycomb.

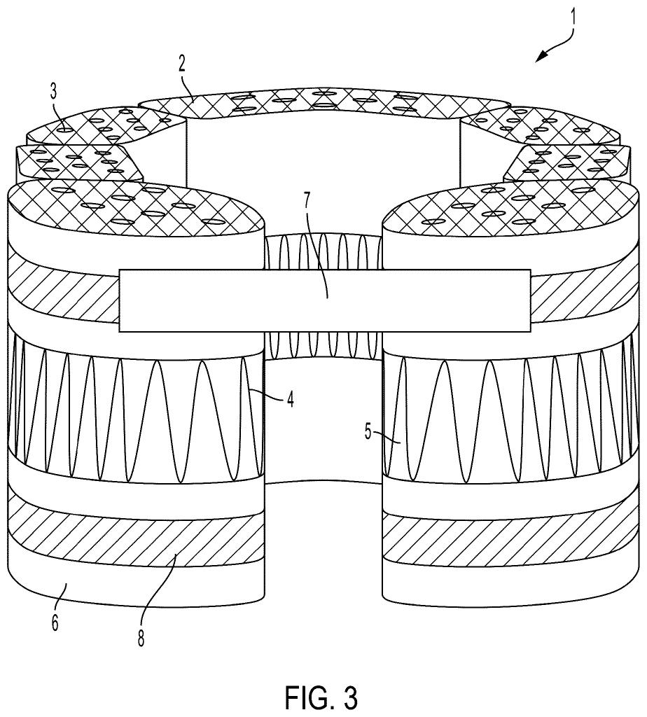

Referring to FIG. 3, an example of neck brace 1 is presented from a front view. In some embodiments, the neck brace 1 contains a fastener 7. In some embodiments, the neck brace 1 also contains a fastener attachment 8. In some examples, the fastener attachment 8 is attached to one or more support segments. In some examples, the fastener 7 could be a snap fastener. In other examples, the fastener 7 may be a hook and loop fastener. Additional examples of fastener 7 include magnetic clasps, buckles, or zipper systems.

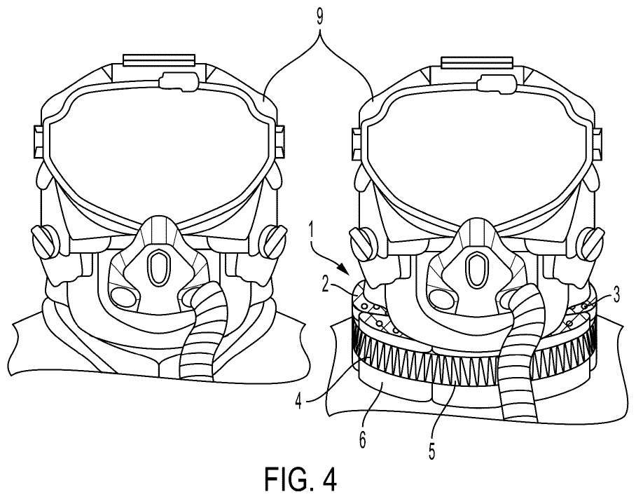

Referring to FIG. 4, an example of neck brace 1 connected to a helmet is presented. In one embodiment, the neck brace 1 can be connected to a helmet. For instance, the neck brace 1 can be permanently connected to a helmet 9. In some embodiments, the neck brace 1 could be an optional attachment to a helmet. In some examples, the neck brace 1 could be an optional attachment to a variety of helmets. For instance, the neck brace 1 could be attached to an aviation helmet, motorcycle helmet, sports helmet (such an American football helmet, cycling helmet, hockey helmet, racecar helmet, military combat helmet, firefighter helmet, or equestrian helmet.

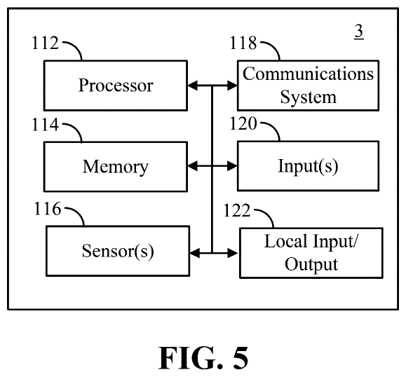

Referring now to FIG. 5, a sample computing device 3 is depicted. In some examples, computing device 3 can include processor 112. In some embodiments, the processor 112 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), etc.

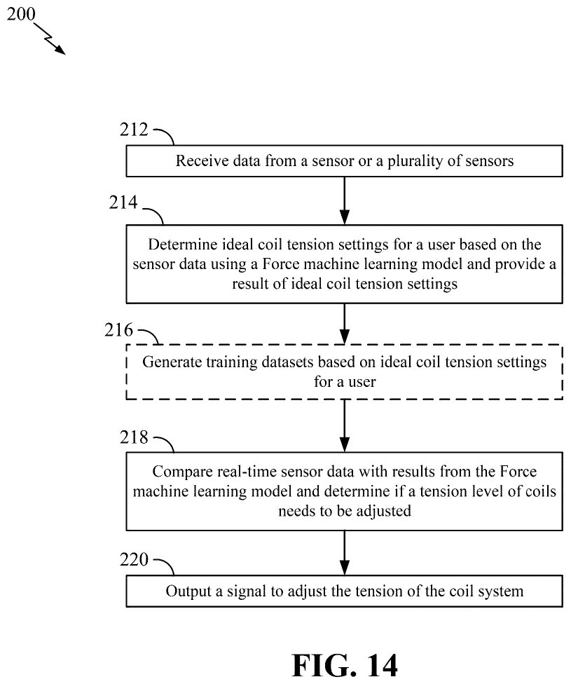

In further examples, computing device 3 can further include a memory 114. The memory 114 can include any suitable storage device or devices that can be used to store suitable data and instructions that can be used, for example, by the processor 112 to receive data corresponding to a sensor 116 or a plurality of sensors 116. For example, the memory 114 could be used to store instructions to adjust the tension of the coils of a particular support segment based on changes in sensor data. The memory 114 can include any suitable volatile memory, non-volatile memory, storage, or any suitable combination thereof. For example, memory 114 can include random access memory (RAM), read-only memory (ROM), electronically-erasable programmable read-only memory (EEPROM), one or more flash drives, one or more hard disks, one or more solid state drives, one or more optical drives, cloud-based resources, etc. In some embodiments, the processor 112 can execute all or at least a portion of processes 200 as described in connection with FIG. 14.

In some embodiments, computing device 3 can further include a sensor 116. In some embodiments the computing device 3 can include a plurality of types of sensors. In some examples, the sensors 116 of computing device 3 can be integrated into the neck brace and can be configured to monitor motion, force, environmental, and/or physiological parameters in real time to enable adaptive control of the support segments during high-speed travel and high force maneuvers. A variety of sensors 116 of computing device 3 can be integrated into the dynamic neck brace. For example, in certain implementations, the sensors may include accelerometers for measuring linear acceleration, gyroscopes for detecting angular velocity and rotational movement, or magnetometers for directional heading. In some embodiments, the sensors may include strain gauge sensors and load cells to measure mechanical strain and applied force. In additional embodiments, the sensors may be able to perform physiological monitoring. For example, physiological monitoring may be achieved through electromyography (EMG) sensors for muscle activity, pulse oximeters for blood oxygen saturation, electrocardiogram (ECG) sensors for cardiac activity, and vascular pressure sensors for blood pressure. Additionally, in some embodiments, the sensors may be vibration sensors that can identify oscillatory forces.

In further examples, computing device 3 can further include communications system 118. For example, communications system 118 can include one or more transceivers, one or more communication chips and/or chip sets, etc. In a more particular example, communications system 118 can include hardware, firmware and/or software that can be used to establish a Wi-Fi connection, a Bluetooth connection, a cellular connection, an Ethernet connection, a local network, etc.

In some examples, the computing device 3 can further transmit an output signal 122 to a series of connected coils 4. The form of output signal 122 may depend upon the data provided by sensors 116. For instance, based on the data received by sensors 116, the memory 114 and processor 112 of computing device 3 could direct an output signal 122 to tighten the series of connected coils 4. For instance, the output signal 122 could direct a mechanism to tighten the coils. For example, a potential mechanism for tightening or compressing the coils could involve a rotary tensioning system integrated into the neck brace 1. In some embodiments, this mechanism may include a dial or knob connected to a threaded rod or spool, which winds or unwinds a tension cable linked to the series of connected coils 4. In some embodiments, rotating the dial would increase or decrease the compression of the series of connected coils 4, allowing precise adjustment of support. In other embodiments, an electromechanical actuator controlled by a microcontroller could automatically tighten the series of connected coils 4 based on sensor data and transmitted to output signal 122.

In further examples, computing device 3 can further include one or more inputs 120. In further embodiments, the input(s) 120 can include any suitable input devices that can be used to receive user input, such as a touchscreen, a phone, a computer, etc. For example, the computing device 3 could receive information from an input to describe the user's preferred level of coil tension at a particular force.

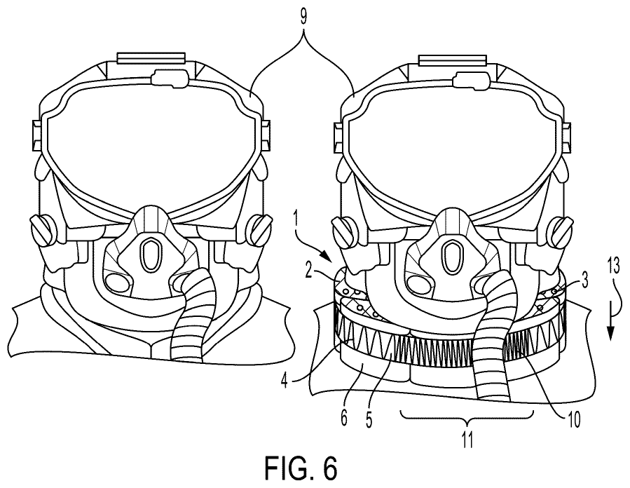

Referring now to FIG. 6, an example of neck brace 1 in response to downward force 13 is presented. For instance, due to the immense speed of a traveling vehicle, such as an airplane, a individual's head will be thrust forward. To counteract the force, such as a downward force 13, the series of connected coils 4 can compress or condense over a single support segment 6 or over multiple support segments. Example embodiment 10 depicts a representative compressed coil. In some embodiments, the compression of the series of connected coils 4 corresponds to a tension level of the series of connected coils. For instance, in some embodiments, if the series of connected coils 4 is uncompressed, this results in a lower tension level. However, in other embodiments, if the series of connected coils 4 is compressed, as depicted in compressed coil 10, the tension of the coil is higher. Example embodiment 11 depicts a compressed coil 10 over multiple supporting segments. For instance, FIG. 6 depicts neck brace 1 wherein the tension level of the series of connected coils adjusts over a series of multiple support segments.

In some embodiments, the coils can be tightened or compressed via a rotary tensioning system integrated into the neck brace 1. In other embodiments, the coils can be tightened or compressed via a dial or knob connected to a threaded rod or spool, which winds or unwinds a tension cable linked to the series of connected coils 4. In some embodiments, rotating the dial would increase or decrease the compression of the coils, allowing precise adjustment of support. In other embodiments, an electromechanical actuator controlled by a microcontroller could automatically tighten the series of connected coils 4 based on sensor 116 data and the output of computing device 3.

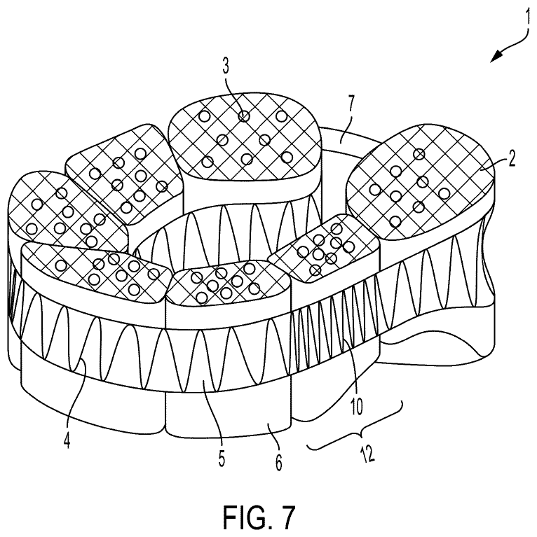

Referring now to FIG. 7, neck brace 1 is depicted from an orthogonal view further depicting an example of compressed coil 10. For instance, FIG. 7 depicts example embodiment 12, illustrating neck brace 1, wherein the compressed coil 10 is compressed in a region corresponding to a single support segment 6 while the series of connected coils 4 is a continuous set of coils that surrounds the one or more support segments 6. In other words, in some embodiments, although the series of connected coils 4 is a continuous set of coils that surrounds all of the support segments 6 in example neck brace 1, only a particular region of the series of connected coils 4 may become compressed coil 10.

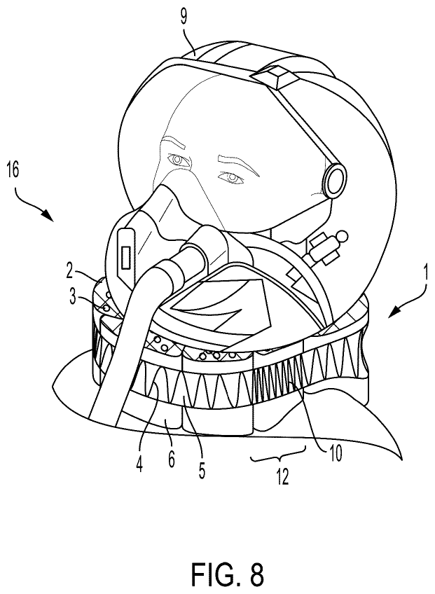

Referring now to FIG. 8, an example of neck brace 1 connected to pilot's helmet 9 in response to a diagonal force 16 is depicted. For example, computing device 3 may provide an output signal 122 to only condense the coils (i.e. adjust the tension of the coils) surrounding a single support segment as depicted by embodiment 12 in response to diagonal force 16.

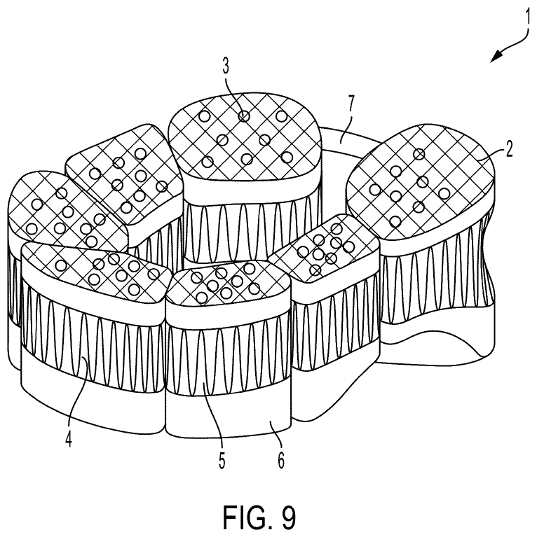

Referring now to FIG. 9, an example of neck brace 1 is depicted whereby the series of connected coils 4 is a single set of coils that corresponds to a single support segment 6. In other words, in some embodiments, the series of connected coils 4 is integrated on the outer and inner surfaces of a single support segment 6. In some examples, the tension of the coils of the series of connected coils 4 is adjusted based on the output of computing system 3 associated with the single support segment 6.

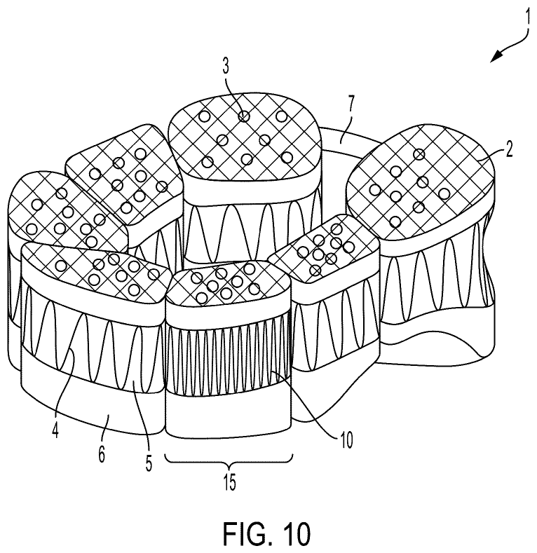

Referring now to FIG. 10, neck brace 1 is depicted from an orthogonal view further depicting an example of compressed coil 10, whereby the compressed coil 10 of the series of connected coils 4 is compressed in a single support segment 6. For instance, FIG. 10 depicts example embodiment 15, illustrating neck brace 1 wherein the compressed coils 10 are compressed in a region corresponding to a single support segment 6 while the series of connected coils 4 is a single set of coils that surrounds one support segment 6. In other words, in some embodiments as depicted in FIG. 10, the series of connected coils 4 that surround each support segment 6 are each an individual unit, rather than one continuous set of coils surrounding the all of the supportive segments 6 (which is the embodiment depicted in FIG. 7).

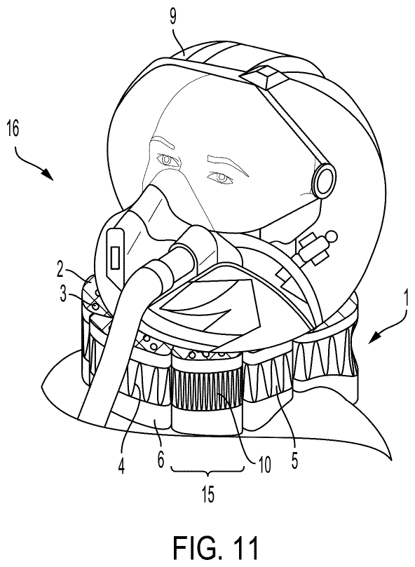

Referring now to FIG. 11, an example of neck brace 1 connected to pilot's helmet 9 in response to a diagonal force 16 is depicted. For example, computing device 3 may provide an output signal 122 to only condense the coils (i.e. adjust the tension of the coils) surrounding a single support segment as depicted by embodiment 15 in response to diagonal force 16.

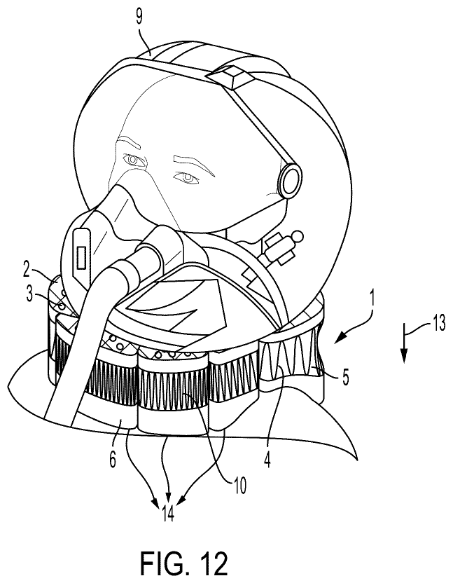

Referring now to FIG. 12, an example of neck brace 1 in response to downward force 13 is presented. Example embodiment 14 depicts multiple supporting segments each with compressed coil 10, whereby each support segment functions as an individual unit with a single set of coils as the series of connected coils 4, and the multiple supporting segments (each functioning as individual units) each have compressed coil 10 in response to downward force 13 based on the output of each individual support segment computing system 3.

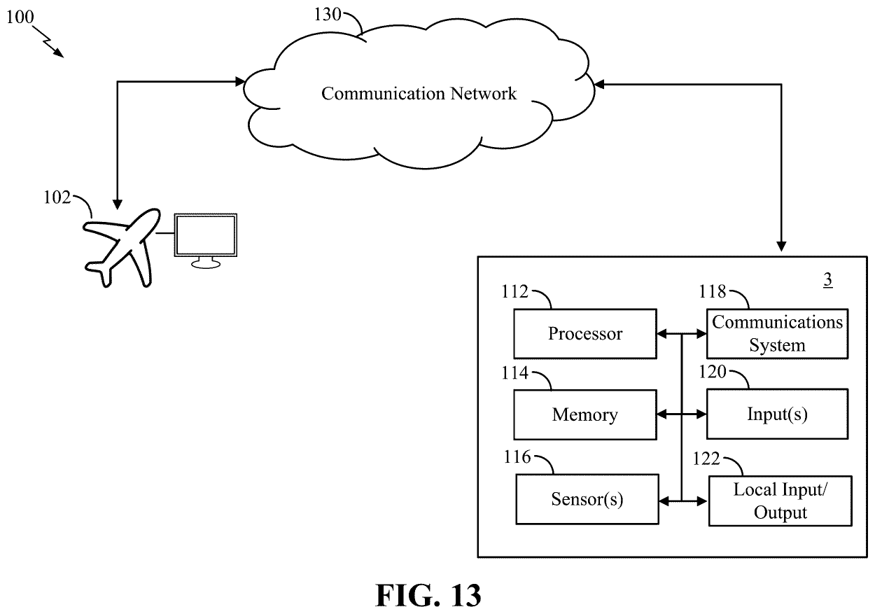

FIG. 13 shows a block diagram illustrating a system for protecting an individual's head and neck from injury, whereby a central computer of the aircraft or vehicle can communicate with the computing device 3 of neck brace 1, according to some embodiments. In some embodiments, computing device 3 can receive or transmit information over a communication network 130. In some examples, the communication network 130 can be any suitable communication network or combination of communication networks. For example, the communication network 130 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 130 can be a local area network, a wide area network, a public network (e.g., the Internet), a private or semi-private network (e.g., a corporate or university intranet), any other suitable type of network, or any suitable combination of networks. Communications links shown in FIG. 13 can each be any suitable communications link or combination of communications links, such as wired links, fiber optic links, Wi-Fi links, Bluetooth links, cellular links, etc. For instance, computing device 3 can receive one or more inputs from a central computer of the aircraft. For example, computing device 3 may receive real-time operational data from the central computer of the aircraft through communication network 130.

For example, in some embodiments, the data exchange between the central computer of the aircraft over communication network 130 enables computing device 3 to anticipate and respond to dynamic flight conditions. For instance, the types of information provided by the aircraft's central computer may include current and projected G-force levels, acceleration vectors, angular velocity, altitude, airspeed, and maneuver profiles such as banking angles or dive trajectories. Additional data may include cabin pressure, temperature, and vibration data from the aircraft's structural sensors. In some embodiments, the computing device 3 can process the external data from the aircraft in conjunction with the data provided by sensors 116 to calculate and determine optimal tension levels of series of connected coils 4 for the support segments.

FIG. 14 is a flow diagram illustrating an example of process 200 for outputting a signal to adjust the tension of the series of connected coils 4. As described below, a particular implementation can omit some or all illustrated features/steps, may be implemented in some embodiments in a different order, and may not require some illustrated features to implement all embodiments. In some examples, an apparatus (e.g., processor 112 with memory 114) in connection with FIG. 5 can be used to perform example process 200. However, it should be appreciated that any suitable apparatus or means for carrying out the operations or features described below may perform process 200.

In some embodiments, process 100 can be used to determine if the tension of the series of connected coils 4 needs to be adjusted. In some examples, process 100 can be used to determine if the tension of the series of connected coils 4 needs to be adjusted. In some examples, process 100 can be used to determine if the tension across the continuous set of coils as one unit across multiple support segments needs to be adjusted. In other examples, process 100 can be used to determine if the tension across a single set of connected coils corresponding to a particular supportive segment needs to be adjusted.

At step 212, process 200 can receive data from a sensor 116 or a plurality of sensors 116. For example, process 200 could receive data corresponding to a particular gyroscope sensor 116 detecting angular velocity and rotational movement.

At step 214, process 200 can determine the ideal coil tension settings for a user based on the sensor data using a Force machine learning model and provide a result of ideal coil tension settings. For example, a user, such as a pilot, could use input 120 to provide a desired coil tension level to computing device 3 for one or more of the support segments 6 to neck brace 1. In other embodiments, process 200 may determine an ideal coil tension setting for a user based on real-time sensor data using a Force machine learning model. For instance, sensor 116 may be a gyroscope that provides real-time information regarding angular velocity and rotational movement to the Force machine learning model for processing and determination of the ideal coil tension settings for the series of connected coils 4 integrated on one or more support segments 6 based on the gyroscope sensor data.

In some embodiments, the Force machine learning model is a supervised learning model. For instance, in some embodiments, a supervised learning model includes regression algorithms, such as linear regression or polynomial regression, configured to predict continuous variables derived from sensor readings. In other implementations, classification algorithms such as decision trees, random forests, and support vector machines may be utilized as learning models to categorize sensor states or detect anomalies.

In other embodiments, the Force machine learning model may be an unsupervised learning model. For instance, an unsupervised learning model may include clustering algorithms, such as K-means or hierarchical clustering, which may be configured to group sensors or operational states based on similarity metrics. Additionally, dimensionality reduction techniques, such as principal component analysis (PCA), may be employed to reduce the complexity of sensor datasets while preserving essential variance. In some examples, the unsupervised machine learning models may enable process 214 to discover hidden structures and optimize sensor configurations without requiring predefined labels.

In other embodiments, the Force machine learning model may utilize deep learning architectures to process complex, high-volume sensor data streams. Convolutional neural networks (CNNs) may be implemented to capture spatial correlations and short-term temporal features within sensor arrays, while recurrent neural networks (RNNs), including long short-term memory (LSTM) units, may be employed to model sequential dependencies in time-series sensor data. In certain implementations, transformer-based architectures may be integrated to perform multimodal sensor fusion, allowing the system to combine heterogeneous sensor inputs for enhanced predictive accuracy. In some embodiments, these deep learning models may be optimized for real-time inference in edge computing environments.

In further embodiments, the Force machine learning model may incorporate reinforcement learning algorithms to enable adaptive decision-making based on sensor feedback. The reinforcement learning framework may include an agent configured to interact with an environment, receiving sensor-derived state information and selecting actions to maximize cumulative rewards. In certain implementations, Q-learning or deep Q-network (DQN) techniques may be employed to optimize control strategies for dynamic systems, such as robotic actuators or smart industrial equipment. This approach allows the Force machine learning model to continuously improve performance through iterative learning, leveraging sensor data to refine operational policies over time.

At step 216, process 200 can optionally generate training datasets derived from the calculated ideal coil tension settings. These datasets may include labeled examples correlating sensor input patterns with corresponding tension outputs. Such datasets may be stored for future model refinement, enabling supervised learning approaches to improve predictive accuracy across diverse user profiles and operational scenarios.

At step 218, process 200 can continuously compare incoming or real-time sensor data with the predicted results from the Force machine learning model to determine whether the current coil tension level at any supportive segment 6 requires adjustment. If the comparison indicates a deviation beyond a predefined threshold based on sensory 116 data, the system may initiate corrective action to restore optimal tension.

At step 220, process 200 can output a signal to adjust the tension of the coil system. For instance, upon determining that an adjustment is necessary, step 220 of process 200 may output a control signal to the coil tensioning mechanism. In some embodiment, this signal may instruct the coil tensioning mechanism to increase or decrease tension in real time, ensuring dynamic adaptation to changing conditions. In some embodiments, this feedback loop operates continuously to maintain user safety and comfort during high-force environments.

What is claimed is:

1. A neck brace comprising:
one or more support segments,
a computing device; and
a series of connected coils integrated on outer and inner surfaces of the one or more supportive segments,
wherein a tension level of the series of connected coils adjusts based on an output from the computing device.

2. The neck brace of claim 1, wherein the computing device comprises a sensor.

3. The neck brace of claim 1, wherein the computing device comprises a plurality of sensors.

4. The neck brace of claim 2, wherein the sensor is selected from the group comprising one or more of: accelerometers, gyroscopes, magnetometers, strain gauge sensors and load cells, electromyography sensors, pulse oximeters, electrocardiogram sensors, vascular pressure sensors, and vibration sensors.

5. The neck brace of claim 1, wherein the one or more support segments comprise an absorptive padding.

6. The neck brace of claim 5, wherein the absorptive padding comprises one or more of: hexagonal gel support, honeycomb cardboard, aluminum honeycomb, polycarbonate, polypropylene, carbon fiber composite, Kevlar-reinforced polymer, thermoplastic elastomers (TPE), foamed polyurethane, titanium alloy honeycomb, and recycled or non-recycled Polyethylene Terephthalate (PET) honeycomb.

7. The neck brace of claim 1, wherein the tension level of the series of connected coils adjusts over multiple supporting segments.

8. The neck brace of claim 1, wherein the tension level of the series of connected coils adjusts over a single support segment.

9. The neck brace of claim 1, wherein the series of connected coils is a continuous set of coils that is integrated on outer and inner surfaces of the one or more supportive segments.

10. The neck brace of claim 1, wherein the series of connected coils is a single set of connected coils corresponding to a particular supportive segment, wherein the series of connected coils is integrated on outer and inner surfaces of the supportive segment.

11. The neck brace of claim 2, wherein the computing device further comprises:
a memory;
a processor communicatively coupled to the memory;
wherein the memory stores a set of instructions, which, when executed by the processor, cause the processor to:
receive data from the sensor;
determine an ideal coil tension setting for a user based on the sensor data using a Force machine learning model;
provide a result of ideal coil tension settings;
compare the result of the ideal coil tension settings with real-time sensor data;
determine if a tension level of coils needs to be adjusted; and
output a signal to adjust the tension level of the series of connected coils.

12. The neck brace of claim 11, wherein the memory further comprises a set of instructions to generate training datasets based on ideal coil tension settings for a user.

13. The neck brace of claim 1, further comprising a cloth.

14. A system for protecting the head or neck from injury, the system comprising:
a neck brace comprising one or more support segments;
a series of connected coils integrated on outer and inner surfaces of the supportive segment;
a computing device comprising:
a sensors;
a memory;
a processor communicatively coupled to the memory;
wherein the memory stores a set of instructions, which, when executed by the processor, cause the processor to:
receive data from the sensor;
determine an ideal coil tension setting for a user based on the sensor data using a Force machine learning model;
provide a result of ideal coil tension settings;
compare the result of the ideal coil tension settings with real-time sensor data;
determine if a tension level of coils needs to be adjusted; and
output a signal to adjust the tension level of the series of connected coils.

15. The system of claim 14, wherein the one or more support segments comprise an absorptive padding.

16. The system of claim 15, wherein the absorptive padding comprises one or more of: hexagonal gel support, honeycomb cardboard, aluminum honeycomb, polycarbonate, polypropylene, carbon fiber composite, Kevlar-reinforced polymer, thermoplastic elastomers (TPE), foamed polyurethane, titanium alloy honeycomb, and recycled or non-recycled Polyethylene Terephthalate (PET) honeycomb.

17. The system of claim 14, wherein the tension level of the series of connected coils adjusts over multiple supporting segments.

18. The system of claim 14, wherein the tension level of the series of connected coils adjusts over a single support segment.

19. The system of claim 14, wherein the series of connected coils is a continuous set of coils that is integrated on outer and inner surfaces of the one or more supportive segments.

20. The system of claim 14, further comprising a cloth.
